(19) United States
(12) Patent Application Publication
(10) Pub. No.: US 2023/0364193 A1
(43) Pub. Date: Nov. 16, 2023

(54) COMBINED DRUG FOR TREATING CORONARIVUS DISEASE 2019

(71) Applicants: SICHUAN UNIVERSITY WEST CHINA HOSPITAL, Sichuan (CN); SICHUAN HUIYANG LIFE SCIENCE & TECHNOLOGY CORP., Sichuan (CN)

(72) Inventors: Lunxu LIU, Sichuan (CN); Chuan LI, Sichuan (CN); Guangwen WEI, Sichuan (CN)

(21) Appl. No.: 18/012,025

(22) PCT Filed: Jun. 21, 2021
(86) PCT No.: PCT/CN2021/101213

§ 371 Date: Dec. 21, 2022

(30) Foreign Application Priority Data
Jun. 22, 2020 (CN) 202010577357.1

(51) Int. Cl. A61K 38/21; A61P 31/14; A61K 9/00; A61K 31/513; A61K 31/427; A61K 31/4045

(52) U.S. Cl. CPC A61K 38/212; A61P 31/14; A61K 9/0073; A61K 31/513; A61K 31/427; A61K 31/4045

(57) ABSTRACT

Disclosed are a combined drug or a kit and the use thereof in the preparation of a drug for treating coronavirus Disease 2019 or Middle East respiratory syndrome. The combined drug or the kit contains rSIFN-co and a baseline therapeutic drug which are administered simultaneously or separately, wherein the baseline therapeutic drug is 1) lopinavir ritonavir, or 2) arbidol. The combined drug can effectively improve the condition of a patient suffering from moderate to severe COVID-19.

### CROSS REFERENCE TO RELATED APPLICATIONS

This application is a National Phase application under 35 U.S.C. 371 of PCT/CN2021/101213 filed Jun. 21, 2021, which claims the benefit of and priority to Chinese Provisional Application No. 202010577357.1 filed Jun. 22, 2020.

### REFERENCE TO SEQUENCE LISTING

The Sequence Listing submitted Jan. 20, 2023, as a text file named “SICH_100_371_ST25.txt” created on Jan. 18, 2023, and having a size of 4,096 bytes is hereby incorporated by reference pursuant to 37 C.F.R. § 1.52(e)(5).

### FIELD OF THE INVENTION

The invention belongs to the field of antiviral drugs.

### BACKGROUND OF THE INVENTION

Coronavirus disease 2019 (COVID-19) is a pneumonia caused by severe acute respiratory syndrome coronavirus 2 (SARS-CoV-2) infection. SARS-CoV-2 is a new type of virus belonging to the coronavirus family. It is the seventh coronavirus pathogen that can infect humans and the third coronavirus pathogen that can cause severe clinical syndrome after SARS-CoV and Middle East Respiratory Syndrome (MERS)-CoV.

COVID-19 broke out in early 2020, and it is still raging around the world. As of Jun. 11, 2020, a total of 7,313,661 cases of COVID-19 have been confirmed worldwide, with a total of 413,854 deaths, posing a great threat to human life and health. On the other hand, due to the high infectiousness of COVID-19, various countries and regions have to adopt very strict control measures, and the global economy has been greatly affected. The even more worrying news is that there may be a large-scale outbreak in autumn and winter since there is currently no drug that can effectively treat COVID-19. Therefore, the importance of developing effective drugs for the treatment of COVID-19 is self-evident.

Researchers have tried a lot of antiviral drugs, including some drugs for treatment of SARS and MERS, but the effects thereof were not desirable. For example, by evaluating the in vitro inhibitory effects of ribavirin, penciclovir, nitazolamide, nafamostat, chloroquine, remdesivir, favipiravir and lopinavir on SARS-CoV-2, it was found that chloroquine, remdesivir, lopinavir, ritonavir, arbidol, etc., showed good effects on SARS-CoV-2 in vitro, but poor results in clinical trials with little therapeutic effect; instead, they were prone to side effects such as serious gastrointestinal reactions and impaired kidney functions, and thus were not suitable for long-term use. At present, there are no relevant reports on drugs with excellent efficacy.

Interferon (IFN) is a low-molecular-weight glycoprotein with similar structure and function produced by the host through antiviral response during the infection of virus. There are 3 main types of interferon: type I interferon, type II interferon and type III interferon. Type I interferon (which can be divided into two classes: α and β) can be used for clinical antiviral therapy. Studies have shown that α-interferon (IFN-α) can be combined with ribavirin, oseltamivir, lopinavir/ritonavir and other antiviral drugs or glucocorticoids to treat SARS and MERS. In the second, third, fourth, fifth, sixth, and seventh editions of the diagnosis and treatment scheme for COVID-19 issued by the National Health Commission of the People's Republic of China, it is proposed that IFN-α be administered by nebulization, at 5 million IU (international unit) plus 2 ml water for injection, twice a day.

Recombinant super-compound interferon (rSIFN-co) is a new type of genetically engineered interferon produced by changing 65 bases of the 60 amino acid genetic code of IFN-α without changing its amino acid composition. rSIFN-co was initially used to fight against SARS and had a good curative effect, but its effect on COVID-19 has not been reported yet.

### SUMMARY OF THE INVENTION

The problem to be solved by the present invention is to provide a combined drug for treating COVID-19.

The present invention provides the following technical solutions:
1. A combined drug or kit for the treatment of COVID-19, which comprises rSIFN-co and a baseline therapeutic drug which are administered simultaneously or separately,
wherein the baseline therapeutic drug is
1) lopinavir/ritonavir, or
2) arbidol.
2. The combined drug or kit as mentioned above, wherein the amount of rSIFN-co in each unit preparation is 5 million to 24 million IU, and the amount of rSIFN-co in each daily dosage is 10 million to 48 million IU.

Preferably, the amount of rSIFN-co in each unit preparation is 10 million to 14 million IU, and the amount of rSIFN-co in each daily dosage is 20 million to 28 million IU.

Further preferably, the amount of rSIFN-co in each unit preparation is 12 million IU, and the amount of rSIFN-co in each daily dosage is 24 million IU.
3. The combined drug or kit as mentioned above, wherein the rSIFN-co is a preparation for nebulization administration, intramuscular injection or subcutaneous injection.
4. The combined drug or kit as mentioned above, wherein the amounts of lopinavir/ritonavir in daily dosage are 800 mg and 200 mg respectively.
5. The combined drug or kit as mentioned above, wherein the amount of arbidol in daily dosage is 600 mg.
6. Use of rSIFN-co and a baseline therapeutic drug in the preparation of a combined drug or kit for the treatment of COVID-19 or Middle East respiratory syndrome, wherein the baseline therapeutic drug is:
1) lopinavir/ritonavir, or
2) arbidol.
7. The use as mentioned above, wherein in the combined drug or kit, the amount of rSIFN-co in each unit preparation is 5 million to 24 million IU, and the amount of rSIFN-co in each daily dosage is 10 million to 48 million IU;

Preferably, the amount of rSIFN-co in each unit preparation is 10 million to 14 million IU, and the amount of rSIFN-co in each daily dosage is 20 million to 28 million IU;

Further preferably, the content of rSIFN-co in each unit preparation is 12 million IU, and the content of rSIFN-co in each daily dosage is 24 million IU.
8. The use as mentioned above, wherein in the combined drug or kit, the rSIFN-co is a preparation for nebulization administration, intramuscular injection or subcutaneous injection.
9. The use as mentioned above, wherein, in the combined drug or kit, the amounts of lopinavir/ritonavir in daily dosage are 800 mg and 200 mg respectively.
10. The use as mentioned above, wherein, in the combined drug or kit, the amount of arbidol in daily dosage is 600 mg.
11. A method for treating COVID-19, comprising administering rSIFN-co and a baseline therapeutic drug to a subject, wherein, preferably, the COVID-19 is in a moderate to severe stage,
wherein the baseline therapeutic drug is
1) lopinavir/ritonavir, or
2) arbidol.
12. The method as mentioned above, wherein the rSIFN-co is administered as follows: the amount of rSIFN-co in each unit preparation is 5 million to 24 million IU, and the amount of rSIFN-co in each daily dosage is 10 million to 48 million IU;

Preferably, the amount of rSIFN-co in each unit preparation is 10 million to 14 million IU, and the amount of rSIFN-co in each daily dosage is 20 million to 28 million IU;

Further preferably, the amount of rSIFN-co in each unit preparation is 12 million IU, and the amount of rSIFN-co in each daily dosage is 24 million IU.
13. The method as mentioned above, wherein the rSIFN-co is administered by nebulization, intramuscular injection or subcutaneous injection.
14. The method as mentioned above, wherein the amounts of lopinavir/ritonavir in daily dosage are 800 mg and 200 mg respectively.
15. The method as mentioned above, wherein the amount of arbidol in daily dosage is 600 mg.
16. A pharmaceutical composition for the treatment of COVID-19, which comprises rSIFN-co, preferably, the amount of rSIFN-co in each unit preparation is 5 million to 24 million IU, more preferably the amount of rSIFN-co in each unit preparation is 10 million to 14 million IU; preferably, the pharmaceutical composition further comprises a baseline therapeutic drug, further preferably, the baseline therapeutic drug is: 1) lopinavir/ritonavir; or 2) arbidol.

### Advantages

Studies have shown that lopinavir/ritonavir or arbidol alone cannot effectively treat COVID-19. The present invention verified the effect of rSIFN-co against SARS-CoV-2 in vitro, and also verified that the combination of rSIFN-co with lopinavir/ritonavir, or with arbidol had a significant efficacy on COVID-19, and its effect was significantly better than the combination of IFN-α with lopinavir/ritonavir, or with arbidol.

Apparently, based on the above content of the present invention and according to common technical knowledge and conventional means in the art, those skilled in the art can further make other modifications, substitutions or changes in various forms without departing from the spirit of the present invention.

The above content of the present invention will be further described in detail below through specific examples. However, this should not be construed as limiting the scope of the present invention to the examples. All technical solutions achieved based on the contents of the present invention belong to the scope of the present invention.

### DETAILED DESCRIPTION OF THE INVENTION

### Example 1 Pharmacodynamic Study of rSIFN-co against SARS-CoV-2 Virus at Different Multiplicity of Infection In Vitro

In order to evaluate the pharmacodynamics of rSIFN-co against SARS-CoV-2 viruses with different multiplicity of infection in vitro, the present inventors conducted the following experiments.

### 1. Materials and Instruments

Test drugs:

| Drug Name | Supplier | Specification | Batch No. |
|---|---|---|---|
| Recombinant super- | Sichuan Huiyang Life Science | 1.3 mg/ml | 20180904 |
| compound interferon | & Technology Corp.. | | |
| (rSIFN-co) | | | |
| Recombinant Human | Sichuan Huiyang Life Science | 30 μg/ml | R0200101 |
| Interferon α2b | & Technology Corp. | (1 ml: 3 million | |
| Injection (Pseudomonas) | | international units) | |

storage method: Stored at 4° C. in dark experimental materials:
1) Cell line: Vero-E6 cells (ATCC® CRL-1586™);
2) Virus strain: SARS-CoV-2 (National Virus Resource Center, deposit number IVCAS 6.7512);
3) Positive control drug: chloroquine;
4) Reagents: DMEM medium (Gibco), fetal bovine serum (Gibco), DMSO (sigma), double-antibiotics, trypsin, etc.;
5) Kit: Cell Counting Kit-8 (CCK-8) (B34304, bimake);
6) Consumables: cell culture plates, pipettes, etc.

Instruments:

Multifunctional microplate reader (Thermo), carbon dioxide incubator (Thermo), etc.

### 1.2 Evaluation of the Inhibitory Effect of rSIFN-co and Recombinant Human Interferon α2b Injection on SARS-CoV-2 Virus

Specifically, the detection of antiviral activity was performed on a Vero-E6 cell model, with triplicate wells for each experiment, repeated for three times. The following steps were performed.
1) Vero-E6 cells were inoculated in a 96-well plate at 1×10⁴ cells per well the day before;
2) The cell state was observed until the confluence reached about 70%˜80%. Chloroquine, rSIFN-co and recombinant human interferon α2b injection (Pseudomonas) were diluted by 2 fold with DMEM medium containing 2% PBS. The medium in the wells was discarded, and 100 μl DMEM medium containing SARS-CoV-2 virus solution (with MOI as 0.005, 0.05 and 0.5, that is, the virus infection dose was 50 PFU, 500 PFU and 5000 PFU) and corresponding concentration of drug was added to each well, meanwhile, a control group (virus group without drugs and normal cell group) was set up; wherein, 8 concentration gradients were set for each drug, with triplicates for each concentration, and the cells were cultured at 37° C., 5% CO₂;
3) Cytopathic effect (CPE) was observed under an inverted microscope every day. Cell viabilities were measured by CCK-8 method when the control group (virus group without drugs) showed obvious cytopathic effect, and the steps were shown as follows: 1/10 volume of Cell Counting Kit-8 (CCK-8) was directly added to the cell culture medium, mixed well, and air bubbles should be avoided. Cells were incubated in an incubator at 37° C. for 1 hour until the color turned into orange. The normal cell group was set as zero, and the absorbance at 450 nm was measured with a multifunctional microplate reader.
4) CPE inhibition rates of the drug at each concentration were calculated.

CPE inhibition rate (%)=(OD450 of drug group-OD450 of virus group without drug)/(OD450 of normal cell group-OD450 of virus group without drug).

At the same time, half effective concentration (EC₅₀) of the drug was calculated. The results were shown in Table 1.

### 1.3 Cytotoxicity Assay of rSIFN-co and Recombinant Human Interferon α2b injection

1) Vero-E6 cells were inoculated in a 96-well plate at 1×10⁴ cells per well the day before;
2) The cell state was observed until the confluence reached about 50%. The rSIFN-co was diluted by 2.17-fold with 2×DMEM medium containing 4% PBS, as the highest concentration, namely 6×10⁸ pg/ml. The recombinant human interferon α2b injection (Pseudomonas) was diluted by 1.5-fold with 2×DMEM medium containing 4% FBS, as the highest concentration, namely 2×10⁷ pg/ml. And then the rSIFN-co and recombinant human interferon α2b injection were respectively serially diluted by 2-fold with DMEM medium containing 2% FBS:

Specifically, the final concentration of each gradient of rSIFN-co was set as: 6×10⁸ pg/ml, 3×10⁸ pg/ml, 1.5×10⁸ pg/ml, 7.5×10⁷ pg/ml, 3.75×10⁷ pg/ml, 1.875×10⁷ pg/ml, and 0 pg/ml, and the final concentration of each gradient of recombinant human interferon α2b injection (Pseudomonas) was set as: 2×10⁷ pg/ml, 1×10⁷ pg/ml, 5×10⁶ pg/ml, 2.5×10⁶ pg/ml, 1.25×10⁶ pg/ml, 6.25×10⁵ pg/ml, and 0 pg/ml;

At the same time, the positive control chloroquine was serially diluted by 2-fold with DMEM medium containing 2% FBS, and 6 concentration gradients were set;
3) The above DMEM medium containing drugs were added to the cell plate at 100 μl/well with 4 replicates for each concentration and each drug; meanwhile, a control group (a group without drug) and a blank group (a group without cells) were set up. The plates were cultured in an incubator at 37° C. and 5% CO₂;
4) 48 hours after adding the drug, 1/10 volume of Cell Counting Kit48 (CCK-8) was directly added to the cell culture medium, mixed well, and air bubbles should be avoided. Cells were incubated in an incubator at 37° C. for 1 hour until the color turned into orange. The blank group was set as zero, and the absorbance at 450 nm was measured with a multifunctional microplate reader, and the survival rate was calculated as follows: survival rate (%)=OD450 of the test group/OD450 of the control group×100%.

At the same time, half cytotoxicity concentration (CC₅₀) of the drug was calculated, and the results were shown in Table 2. CC₅₀/EC₅₀ was calculated as therapeutic index TI, and the results were shown in Table 3.

TABLE 1
The half effective concentration (EC₅₀) of rSIFN-co and recombinant human interferon
α2b injection (Pseudomonas) in inhibiting the replication of SARS-COV-2
virus with different multiplicity of infection in Vero-E6 cell model

| | multiplicity | | | | | | | | |
|---|---|---|---|---|---|---|---|---|---|
| | of infection | | | | | | | | |
| | (MOI)/infective | EC₅₀ | | | | | | | |
| Drugs | dose (PFU) | Experiment 1 | | Experiment 2 | | Experiment 3 | | means | |
| Recombinant super- | MOI: 0.005 | 15.31 | pg/ml | 14.01 | pg/ml | 10.58 | pg/ml | 13.30 | pg/ml |
| compound interferon | (50 PFU) | | | | | | | | |
| (rSIFN-co) | | | | | | | | | |
| Recombinant Human | | 135.40 | pg/ml | 152.50 | pg/ml | 116.70 | pg/ml | 134.87 | pg/ml |
| Interferon α2b | | | | | | | | | |
| Injection (Pseudomonas) | | | | | | | | | |
| Chloroquine | | 1.70 | μM | 1.77 | μM | 2.34 | μM | 1.94 | μM |
| Recombinant super- | MOI: 0.05 | 141.80 | pg/ml | 125.70 | pg/ml | 102.40 | pg/ml | 123.30 | pg/ml |
| compound interferon | (500 PFU) | | | | | | | | |
| (rSIFN-co) | | | | | | | | | |
| Recombinant Human | | 563.50 | pg/ml | 587.80 | pg/ml | 585.60 | pg/ml | 578.97 | pg/ml |
| Interferon α2b | | | | | | | | | |
| Injection (Pseudomonas) | | | | | | | | | |
| Chloroquine | | 3.65 | μM | 4.23 | μM | 2.34 | μM | 3.41 | μM |
| Recombinant super- | MOI: 0.5 | 188.60 | pg/ml | 139.70 | pg/ml | 125.30 | pg/ml | 151.20 | pg/ml |
| compound interferon | (5000 PFU) | | | | | | | | |
| (rSIFN-co) | | | | | | | | | |
| Recombinant Human | | 2410.00 | pg/ml | 2455.00 | pg/ml | 2060.00 | pg/ml | 2308.33 | pg/ml |
| Interferon α2b | | | | | | | | | |
| Injection (Pseudomonas) | | | | | | | | | |
| Chloroquine | | 5.25 | μM | 5.72 | μM | 5.04 | μM | 5.34 | μM |

TABLE 2
The half cytotoxicity concentration (CC₅₀) of rSIFN-co and recombinant
human interferon α2b injection (Pseudomonas) on Vero-E6 cells

| | CC₅₀ | | | | | | | |
|---|---|---|---|---|---|---|---|---|
| drugs | Experiment 1 | | Experiment 2 | | Experiment 3 | | means | |
| Recombinant super- | 6.51 × 10⁸ | pg/ml | 8.06 × 10⁸ | pg/ml | 6.80 × 10⁸ | pg/ml | 7.12 × 10⁸ | pg/ml |
| compound interferon | | | | | | | | |
| (rSIFN-co) | | | | | | | | |
| Recombinant Human | 1.43 × 10⁷ | pg/ml | 1.30 × 10⁷ | pg/ml | 1.37 × 10⁷ | pg/ml | 1.37 × 10⁷ | pg/ml |
| Interferon α2b | | | | | | | | |
| Injection (Pseudomonas) | | | | | | | | |
| Chloroquine | 44.51 | μM | 76.25 | μM | 63.36 | μM | 61.37 | μM |

TABLE 3
The therapeutic indexes TI of rSIFN-co and recombinant
human interferon α2b injection (Pseudomonas)

| | multiplicity | | | | | |
|---|---|---|---|---|---|---|
| | of infection | | | | | |
| | (MOI)/infective | | | | | TI |
| Drugs | dose (PFU) | CC₅₀ | | EC₅₀ | | (CC₅₀/EC₅₀) |
| Recombinant super- | MOI: 0.005 | 7.12 × 10⁸ | pg/ml | 13.30 | pg/ml | 5.35 × 10⁷ |
| compound interferon | (50 PFU) | | | | | |
| (rSIFN-co) | | | | | | |
| Recombinant Human | | 1.37 × 10⁷ | pg/ml | 134.87 | pg/ml | 1.02 × 10⁵ |
| Interferon α2b | | | | | | |
| Injection (Pseudomonas) | | | | | | |
| Chloroquine | | 61.37 | μM | 1.94 | μM | 31.63 |
| Recombinant super- | MOI: 0.05 | 7.12 × 10⁸ | pg/ml | 123.30 | pg/ml | 5.77 × 10⁶ |
| compound interferon | (500 PFU) | | | | | |
| (rSIFN-co) | | | | | | |
| Recombinant Human | | 1.37 × 10⁷ | pg/ml | 578.97 | pg/ml | 2.37 × 10⁴ |
| Interferon α2b | | | | | | |
| Injection (Pseudomonas) | | | | | | |
| Chloroquine | | 61.37 | μM | 3.41 | μM | 18.00 |
| Recombinant super- | MOI: 0.5 | 7.12 × 10⁸ | pg/ml | 151.20 | pg/ml | 4.71 × 10⁶ |
| compound interferon | (5000 PFU) | | | | | |
| (rSIFN-co) | | | | | | |
| Recombinant Human | | 1.37 × 10⁷ | pg/ml | 2308.33 | pg/ml | 5.94 × 10³ |
| Interferon α2b | | | | | | |
| Injection (Pseudomonas) | | | | | | |
| Chloroquine | | 61.37 | μM | 5.34 | μM | 11.50 |

The results showed that: in the Vero-E6 cell model, when the initial multiplicity of infection MOI were 0.005, 0.05 and 0.5, the half effective concentration (EC₅₀) of positive control chloroquine in inhibiting the replication of SARS-CoV-2 virus were 1.94, 3.41 and 5.34 μM respectively; the half cytotoxicity concentration (CC₅₀) for Vero-E6 cells was 61.37 μM, and when the virus multiplicity of infection MOI were 0.005, 0.05 and 0.5, the therapeutic indexes (TI) of chloroquine were 31.63, 18.00 and 11.50, respectively.

In the Vero-E6 cell model, when the initial multiplicity of infection MOI were 0.005, 0.05 and 0.5, the half effective concentration (EC₅₀) of recombinant human interferon α2b injection (Pseudomonas) in inhibiting the replication of SARS-CoV-2 virus were 134.87, 578.97 and 2308.33 pg/ml, respectively. The half cytotoxicity concentration (CC₅₀) of recombinant human interferon α2b injection (Pseudomonas) to Vero-E6 cells was 1.37×10⁷ pg/ml. When the virus multiplicity of infection MOI were 0.005, 0.05 and 0.5, the therapeutic indexes (TI) of recombinant human interferon α2b injection (Pseudomonas) were 1.02×10⁵, 2.37×10⁴ and 5.94×10³, respectively.

Recombinant super-compound interferon (rSIFN-co) could inhibit SARS-CoV-2 replication in Vero-E6 cell model in a dose-dependent manner

When the initial multiplicity of infection (MOI) were 0.005, 0.05 and 0.5, the half effective concentration (EC₅₀) of rSIFN-co in inhibiting virus replication were 13.30, 123.30 and 151.20 pg/ml, respectively, showing strong anti-SARS-CoV-2 activity. The half cytotoxicity concentration (CC₅₀) of rSIFN-co to Vero-E6 cells was 7.12×10⁸ pg/ml, showing low cytotoxicity. When the initial multiplicity of infection MOI were 0.005, 0.05 and 0.5, the therapeutic indexes (TI) of rSIFN-co were 5.35×10⁷, 5.77×10⁶ and 4.71×10⁶, respectively, which were much higher than those of the positive control chloroquine, and higher than those of recombinant human interferon αm2b.

To sum up, in vitro tests showed that rSIFN-co had a strong anti-SARS-CoV-2 activity, low cytotoxicity, excellent therapeutic index, and had the potential as a therapeutic drug for diseases caused by SARS-CoV-2 virus.

### Example 2: Phase 2 Clinical Trial of rSIFN-co in the Treatment of COVID-19

From Feb. 10, 2020 to Apr. 5, 2020, the inventors conducted a multi-center, randomized, controlled, single-blinded, phase 2 clinical trial.

The rSIFN-co used in the trial was obtained from Sichuan Huiyang Life Science & Technology Corp., and the IFN-α was obtained from Tianjin Hualida Bioengineering Co., Ltd.

The nucleotide sequence encoding rSIFN-co (SEQ ID NO.1) was as follows:

| | |
|---|---|
| atgtgtgatt tacctcaaac tcattctctt ggtaaccgtc gcgctctgat tctgctggca | 60 |
| cagatgcgtc gtatttcccc gtttagctgc ctgaaagacc gtcacgactt cggctttccg | 120 |
| caagaagagt tcgatggcaa ccaattccag aaagctcagg caatctctgt actgcacgaa | 180 |
| atgatccaac agaccttcaa cctgttttcc actaaagaca gctctgctgc ttgggacgaa | 240 |
| agcttgctgg agaagttcta cactgaactg tatcagcagc tgaacgacct ggaagcatgc | 300 |
| gtaatccagc aagttcgtgt agaagagact ccgctgatga acgtcgactc tattctggca | 360 |
| gttaaaaagt acttccagcg tatcactctg tacctgaccg aaaagaaata ttctccgtgc | 420 |
| gcttgggaag tagttcgcgc tgaaattatg cgttctttct ctctgtctac taacctgcag | 480 |
| gagcgtctgc gccgtaaaga ataatag | 507 |

The amino acid sequence of rSIFN-co (SEQ ID NO.2) was as follows:

MCDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEAC
VIQEVGVEETPLMNVDSILAVKKYFQRITLYLTEKKYSPCAWEVVRAEIM
RSFSLSTNLQERLRRKE

Previous studies showed that administration of rSIFN-co in large dosage was safe, and each dosage could be >10 million IU; however, IFN-α was prone to lead to side effects and could not be used in large dosage, and thus should be administrated at the dosage described in the diagnosis and treatment scheme for COVID-19 issued by the National Health Commission of the People's Republic of China.

Specific methods: A total of 102 patients with moderate or severe COVID-19 were recruited, and 94 subjects in a safety analysis set were randomly divided into rSIFN-co group (46 subjects) and IFN-α group (48 subjects). While receiving baseline treatment, subjects were administrated rSIFN-co (12 million IU) or IFN-α (5 million IU) by nebulization, twice a day. The main endpoint of research was disease remission by day 28, including clinical remission time, imaging inflammation absorption time, time for viral nucleic acid to become negative and clinical remission rate.

The aforementioned baseline treatment means: treatment with lopinavir/ritonavir (trade name Kaletra) or Arbidol. Lopinavir/ritonavir were administrated orally, 2 capsules each time (wherein each capsule contained 200 mg lopinavir and 50 mg ritonavir), twice a day, and the course of treatment did not exceed 10 days; Arbidol was administrated orally, 200 mg each time, three times a day. The course of treatment did not exceed 10 days.

In the two groups, the numbers of subjects receiving baseline treatment (lopinavir/ritonavir, or abidol) were: 22 and 24 subjects in the rSIFN-co group, and 20 and 28 subjects in the IFN-α group, p=0.548; the difference was not statistically significant, and the proportions of the patients of the two groups receiving baseline treatment were the same.

The criteria for imaging inflammation absorption was as follows: based on changes of ground-glass opacities and consolidation areas in the lungs of patients compared with the baseline chest CT, the CT results were graded by two independent radiologists to judge the inflammation absorption.

The standard for viral nucleic acid to turn negative was: real-time fluorescence quantitative PCR detections of two consecutive nasopharyngeal swabs, sputum or lower respiratory tract secretions were negative for SARS-CoV-2 nucleic acid, and the sampling interval between the two detections was greater than 24 hours.

### Result

Overall, referring to Table 4, the rSIFN-co group had a shorter period for clinical remission (median 11.5 days vs 14.0 days, p=0.019), a faster absorption of inflammation (median 8.0 days vs 10.0 days; p=0.002), and a shorter period for viral nucleic acid to turn negative (median 7.0 days vs 10.0 days; p=0.018) as compared to the IFN-α group. On day 28, the clinical remission rate of the rSIFN-co group was significantly higher than that of the IFN-α group (93.5% vs 77.1%). Adverse reactions in the two groups were generally mild. There was no serious adverse reaction in the rSIFN-co group, and 1 case of serious adverse reaction (respiratory failure) in the IFN-α group.

TABLE 4
Results of Clinical Trial Study

| | | | |
|---|---|---|---|
| | rSIFN-co | IFN- α | |
| characteristics | (n = 46) | (n = 48) | differences |

7-level scale on day 7

| | | | | |
|---|---|---|---|---|
| 2: Not hospitalized, but unable | 6 | (13.0) | 6 | (12.5) |
| to return to normal activities | | | | |
| 3: Hospitalized, no need for | 9 | (19.6) | 12 | (25.0) |
| supplemental oxygen | | | | |
| 4: Hospitalized, requiring | 23 | (50.0) | 21 | (43.8) |
| supplemental oxygen | | | | |
| 5: Hospitalized, requiring nasal | 8 | (17.4) | 8 | (16.7) |
| high-flow oxygen therapy or non- | | | | |
| invasive mechanical ventilation | | | | |

| | | | |
|---|---|---|---|
| 6: Hospitalized, requiring | 0 | 1 | (2.0) |
| extracorporeal membrane | | | |
| oxygenation, invasive | | | |
| mechanical ventilation, or both | | | |

7-level scale on day 14

| | | | | |
|---|---|---|---|---|
| 2: Not hospitalized, but unable | 27 | (58.7) | 21 | (43.8) |
| to return to normal activities | | | | |
| 3: Hospitalized, no need for | 10 | (21.7) | 15 | (31.2) |
| supplemental oxygen | | | | |
| 4: Hospitalized, requiring | 8 | (17.4) | 8 | (16.7) |
| supplemental oxygen | | | | |
| 5: Hospitalized, requiring nasal | 1 | (2.2) | 3 | (6.3) |
| high-flow oxygen therapy or non- | | | | |
| invasive mechanical ventilation | | | | |

| | | | |
|---|---|---|---|
| 6: Hospitalized, requiring | 0 | 1 | (2.0) |
| extracorporeal membrane | | | |
| oxygenation, invasive | | | |
| mechanical ventilation, or both | | | |

7-level scale on day 28

| 2: Not hospitalized, but unable | 44 | (95.6) | 44 | (91.6) | | |
|---|---|---|---|---|---|---|
| to return to normal activities | | | | | | |
| 3: Hospitalized, no need for | 1 | (2.2) | 1 | (2.1) | | |
| supplemental oxygen | | | | | | |
| 4: Hospitalized, requiring | 1 | (2.2) | 2 | (4.2) | | |
| supplemental oxygen | | | | | | |
| 5: Hospitalized, requiring nasal | 0 | | 0 | | | |
| high-flow oxygen therapy or non- | | | | | | |
| invasive mechanical ventilation | | | | | | |
| 6: Hospitalized, requiring | 0 | | 1 | (2.1) | | |
| extracorporeal membrane | | | | | | |
| oxygenation, invasive | | | | | | |
| mechanical ventilation, or both | | | | | | |
| Time for clinical | 11.5 | (9.3-16.0) | 14.0 | (10.0-18.0) | 1.76 | (1.10-2.81) |
| improvement (days) | | | | | | |

clinical improvement rate

| | | | | | | |
|---|---|---|---|---|---|---|
| day 7 | 5 | (10.9) | 3 | (6.3) | 4.6 | (−0.07-0.16) |
| day 14 | 30 | (65.2) | 19 | (39.6) | 25.6 | (0.06-0.45) |
| day 28 | 43 | (93.5) | 37 | (77.1) | 16.4 | (0.03-0.30) |
| Time for radiographic | 8.0 | (6.0-8.3) | 10.0 | (7.0-13.0) | 2.19 | (1.32-3.62) |
| improvement (days) | | | | | | |

Radiographic Improvement Rate

| | | | | | | |
|---|---|---|---|---|---|---|
| Day 7 | 20 | (43.5) | 13 | (25.0) | 18.5 | (−0.03-0.35) |
| Day 14 | 39 | (84.8) | 32 | (66.7) | 18.1 | (0.01-0.35) |
| Day 28 | 42 | (91.3) | 38 | (79.2) | 12.1 | (−0.02-0.26) |
| Time for Viral nucleic acid | 7.0 | (5.0-13.0) | 10.0 | (6.3-16.8) | 1.74 | (1.10-2.74) |
| to turn negative (days) | | | | | | |

Rate for Viral nucleic acid to turn negative

| | | | | | | |
|---|---|---|---|---|---|---|
| Day 7 | 23 | (50.0) | 15 | (31.3) | 18.7 | (−0.01-0.38) |
| Day 14 | 35 | (76.1) | 31 | (64.6) | 11.5 | (−0.07-0.30) |
| Day 28 | 45 | (97.8) | 41 | (85.4) | 12.4 | (0.02-0.23) |

| Mortality at Day 28 | 0 | 0 | | — | |
|---|---|---|---|---|---|
| Deterioration rate | 0 | 1 | (2.1) | −2.1 | (−0.08-0.04) |

The above data were n (%) or median (IQR). Hazard ratios for time to events were estimated by Cox models. Differences were shown as the overall rate of clinical improvement, chest CT scan radiographic improvement, and viral nucleic acid turning to negative on days 7, 14, and 28, and rate differences and 95% confidence intervals for deterioration rates.

The results showed that, compared with the group administrated by adding IFN-α, the group administrated by adding rSIFN-co in the baseline treatment improved the condition of patients with moderate to severe COVID-19 faster and more significantly.

The existing clinical randomized controlled trials and large-sample retrospective analysis results showed that the baseline therapeutic drug (lopinavir/ritonavir or arbidol) alone was ineffective for the treatment of COVID-19; therefore, it could be reasonably concluded that the combined administration of rSIFN-co and the above baseline therapeutic drug achieved a significant and effective therapeutic effect on COVID-19, and such antiviral effects mainly resulted from the synergistic effect of interferon and the baseline therapeutic drug. Therefore, the present invention provides a combined drug for the treatment of COVID-19, a method for the treatment of COVID-19, and a pharmaceutical composition containing rSIFN-co for the treatment of COVID-19.

In conclusion, rSIFN-co can be combined with lopinavir/ritonavir or arbidol to prepare a combined drug for the treatment of COVID-19 with good clinical effect, which is of great significance for the prevention and treatment of COVID-19.

1. A combined drug or kit for the treatment of COVID-19, which comprises rSIFN-co and a baseline therapeutic drug which are administered simultaneously or separately,
wherein the baseline therapeutic drug is
a) lopinavir/ritonavir, or
b) arbidol.

2. The combined drug or kit of claim 1, wherein the amount of rSIFN-co in each unit preparation is 5 million to 24 million IU, and the amount of rSIFNco in each daily dosage is 10 million to 48 million IU.

3. The combined drug or kit of claim 1, wherein in the combined drug or kit, the rSIFN-co is a preparation for nebulization administration, intramuscular injection or subcutaneous injection.

4. The combined drug or kit of claim 1, wherein the amounts of lopinavir and ritonavir in daily dosage are 800 mg and 200 mg respectively.

5. The combined drug or kit of claim 1, wherein the amount of arbidol in daily dosage is 600 mg.

6. A method of treating COVID-19, comprising administering rSIFN-co and a baseline therapeutic drug to a subject, wherein the baseline therapeutic drug is:
a) lopinavir/ritonavir, or
b) arbidol.

7. The method of claim 6, wherein the sSIFN-co is administered as follows: the amount of rSIFN-co in each unit preparation is 5 million to 24 million IU, and the amount of rSIFN-co in each daily dosage is 10 million to 48 million.

8. The method of claim 6, wherein the rSIFN-co is administered by nebulization, intramuscular injection or subcutaneous injection.

9. The method of claim 6, wherein the amounts of lopinavir and ritonavir in a daily dosage are 800 mg and 200 mg respectively.

10. The method of claim 6, wherein the amount of arbidol in a daily dosage is 600 mg.

11. A pharmaceutical composition for the treatment of COVID-19, which comprises rSIFN-co.

12. The pharmaceutical composition of claim 11, wherein the amount of rSIFN-co in each unit preparation is 5 million to 24 million IU.

13. The pharmaceutical composition of claim 11, wherein the amount of rSIFN-co in each unit preparation is 10 million to 14 million IU.

14. The pharmaceutical composition of claim 11, further comprising a baseline therapeutic drug.

15. The pharmaceutical composition of claim 14, wherein the baseline therapeutic drug is: a) lopinavir/ritonavir; or b) arbidol.

16. The combined drug or kit of claim 1, wherein the amount of rSIFN-co in each unit preparation is 10 million to 14 million IU, and the amount of rSIFN-co in each daily dosage is 20 million to 28 million IU.

17. The combined drug or kit of claim 1, wherein the amount of rSIFN-co in each unit preparation is 12 million IU, and the amount of rSIFN-co in each daily dosage is 24 million I.

18. The method of claim 7, wherein the amount of rSIFN-co in each unit preparation is 10 million to 14 million IU, and the amount of rSIFN-co in each daily dosage is 20 million to 28 million IU.

19. The method of claim 7, wherein the amount of rSIFN-co in each unit preparation is 12 million IU, and the amount of rSIFN-co in each daily dosage is 24 million IU.

20. The method of claim 6, wherein the COVID-19 is in a moderate to severe stage,


SEQUENCE LISTING (2 sequences)

SEQ ID NO: 1 - DNA, 507 bp, Artificial Sequence
  Synthetic sequence for rSIFN-co
atgtgtgatttacctcaaactcattctcttggtaaccgtcgcgctctgattctgctggcacagatgcgtcgtatttccccgtttagctgcctgaaagaccgtcacgacttcggctttccgcaagaagagttcgatggcaaccaattccagaaagctcaggcaatctctgtactgcacgaaatgatccaacagaccttcaacctgttttccactaaagacagctctgctgcttgggacgaaagcttgctggagaagttctacactgaactgtatcagcagctgaacgacctggaagcatgcgtaatccaggaagttggtgtagaagagactccgctgatgaacgtcgactctattctggcagttaaaaagtacttccagcgtatcactctgtacctgaccgaaaagaaatattctccgtgcgcttgggaagtagttcgcgctgaaattatgcgttctttctctctgtctactaacctgcaggagcgtctgcgccgtaaagaataatag

SEQ ID NO: 2 - protein, 166 aa, Artificial Sequence
  rSIFN-co Synthetic amino acid sequence
MCDLPQTHSLGNRRALILLAQMRRISPFSCLKDRHDFGFPQEEFDGNQFQKAQAISVLEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE